(19) United States
(12) Patent Application Publication
(10) Pub. No.: US 2021/0017337 A1
(43) Pub. Date: Jan. 21, 2021

(54) AROMATIC BISMALEIMIDE COMPOUND, PRODUCTION METHOD THEREOF, AND HEAT-CURABLE CYCLIC IMIDE RESIN COMPOSITION CONTAINING THE COMPOUND

(71) Applicant: Shin-Etsu Chemical Co., Ltd., Tokyo (JP)

(72) Inventors: Yoshihiro TSUTSUMI, Annaka-shi (JP); Yuki KUDO, Annaka-shi (JP); Hiroyuki IGUCHI, Annaka-shi (JP); Atsushi TSUURA, Annaka-shi (JP)

(73) Assignee: Shin-Etsu Chemical Co., Ltd., Tokyo (JP)

(21) Appl. No.: 16/922,481

(22) Filed: Jul. 7, 2020

(30) Foreign Application Priority Data
Jul. 19, 2019 (JP) 2019-133486
Aug. 1, 2019 (JP) 2019-141962

(51) Int. Cl. C08G 73/12; C07D 207/452; C08G 73/10

(52) U.S. Cl. CPC C08G 73/121; C07D 207/452; C08G 2150/00; C08G 2170/00; C08G 73/1064

(57) ABSTRACT

Provided are a novel aromatic bismaleimide compound capable of being turned into a film without using a film-forming agent, and dissolved even in a solvent other than a high-boiling aprotic polar solvent; a production method of such compound; and a heat-curable cyclic imide resin composition that contains such compound, and is capable of being cured at a low temperature and turned into a cured product superior in mechanical properties, heat resistance, relative permittivity, dielectric tangent, moisture resistance and adhesiveness. The aromatic bismaleimide compound is represented by the following formula (1):

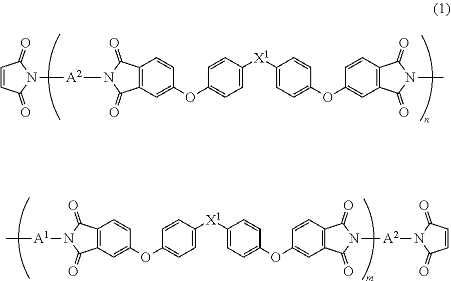

wherein X1 independently represents a divalent group, m represents a number of 1 to 30, n represents a number of 1 to 5, each of A¹ and A² independently represents a divalent aromatic group. The heat-curable cyclic imide resin composition contains the above compound as a component (A), a reaction initiator (B) and an organic solvent (C).

### BACKGROUND OF THE INVENTION

### Field of the Invention

The present invention relates to an aromatic bismaleimide compound, a production method thereof, and a heat-curable cyclic imide resin composition containing such compound.

### Background Art

Bismaleimide resin is known as one of high heat-resistant resins, and has been considered as having a possibility of filling a gap between epoxy resins and polyimides in terms of heat resistance. In recent years, reports have been made on novel bismaleimide compounds (JP-A-2011-219539 and JP-A-2018-012671). Further, reports have also been made on a bismaleimide compound having an extremely low dielectric property (JP-A-2014-194021). These compounds are mainly used as resins for substrates, and are widely used in, for example, impregnating varnishes, laminated sheets or even molded products. However, in most cases, since a bismaleimide compound itself cannot be turned into a film without the aid of a film-forming agent, characteristics unique to a bismaleimide compound may not be efficiently utilized.

Most bismaleimide compounds are low-molecular compounds having a molecular weight of not higher than 2,000; or monomers. While there is known a bismaleimide compound having a high molecular weight by containing maleimide in its repeating units (JP-A-2012-036233), only an extremely small number of cases have been reported on a high-molecular weight bismaleimide compound having a linear chain-like or chainlike high-molecular backbone in the main chain of the molecule, and having maleimide groups at both ends of the molecule.

Further, most aromatic bismaleimide compounds have a fault of, for example, only being able to be dissolved in high-boiling aprotic polar solvents such as NMP (N-methyl-2-pyrolidone) and DMAc (N,N-dimethylacetamide); desired are aromatic bismaleimide compounds capable of being dissolved in versatile solvents other than high-boiling aprotic polar solvents.

Further, in recent years, digital signals with higher frequencies have become increasingly prevalent to match a higher data-processing speed and a larger capacity of a high functional mobile terminal such as a smartphone and a tablet computer. Printed wiring layout for signal transmission is critical to achieve a higher performance of such high-frequency electronic part. That is, a signal transmission speed needs to be raised to a higher level without impairing the quality of a high-speed digital signal having a high-level frequency.

Here, smaller relative permittivity and dielectric tangent are required to reduce the transmission loss of a high-frequency digital signal. Thus, an extremely low relative permittivity and dielectric tangent are required in various materials for use in high-frequency electronic parts such as a printed-wiring board for a high functional mobile terminal or the like of recent years.

In this regard, there has been reported a polyimide resin having a low dielectric property (JP-A-2013-199646 and JP-A-2016-069651).

Since a polyimide resin is superior in heat resistance, flame retardancy, mechanical properties, electrical insulation property and the like, it is widely used as a varnish for an interlayer insulation film or surface protective film of a semiconductor. As previously disclosed, a polyimide resin in the state of a varnish may be applied to a semiconductor element or the like either directly or via an insulation film, followed by curing the same so as to form a protective film made of polyimide resin, and then performing encapsulation with a molding material such as an epoxy resin (JP-A-2007-008977 and JP-A-2010-070645). Further, a polyimide resin may also be used as a film after removing a solvent from the varnish (JP-A-2018-134808).

Such polyimide varnish is usually produced by dissolving polyimide in N-methyl-2-pyrolidone (NMP). While NMP has long been used as an aprotic polar solvent in numerous situations, a stricter restriction is now imposed on the usage thereof mostly and particularly in Europe as the solvent has a high boiling point and toxicities. Further, since an extremely high temperature of not lower than 250° C. is required to cure polyimide, a substitute material is desired.

### SUMMARY OF THE INVENTION

Therefore, it is an object of the present invention to provide a novel aromatic bismaleimide compound capable of being turned into a film without using a film-forming agent, and dissolved even in a solvent other than a high-boiling aprotic polar solvent; and a production method of such compound.

Further, another object of the present invention is to provide a heat-curable cyclic imide resin composition capable of being cured at a low temperature without using an aprotic polar solvent such as NMP, and turned into a cured product superior in mechanical properties, heat resistance, relative permittivity, dielectric tangent, moisture resistance and adhesiveness; an adhesive agent, a substrate material, a primer and a coating material each using the above composition; and a semiconductor device having a cured product of the above composition.

The inventors of the present invention diligently conducted a series of studies to solve the aforementioned problems, and completed the invention as follows. That is, the inventors found that the aromatic bismaleimide compound described below as well as a heat-curable cyclic imide resin composition containing such compound could achieve the above objectives.

[1]

An aromatic bismaleimide compound represented by the following formula (1):

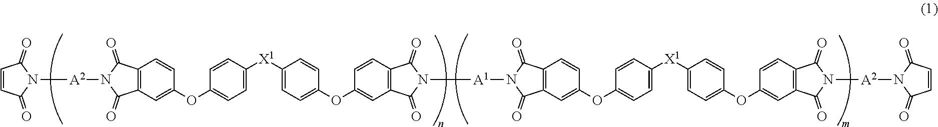

wherein X¹ independently represents a divalent group, each of A¹ and A² independently represents a divalent aromatic group, m represents a number of 1 to 30, n represents a number of 1 to 5,

the divalent group represented by X¹ being selected from groups expressed by the following formulae:

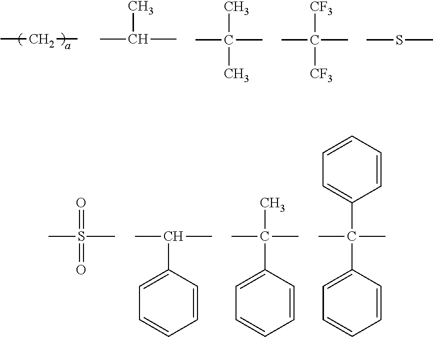

wherein a represents a number of 1 to 6,

the divalent aromatic group represented by each of A¹ and A² being expressed by the following formula (2) or (3):

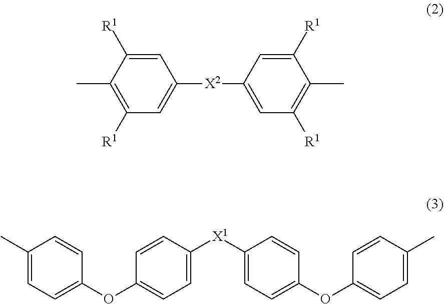

wherein X¹ is defined as above, X² independently represents a divalent group, R¹ independently represents a hydrogen atom, a chlorine atom, or a substituted or unsubstituted aliphatic hydrocarbon group having 1 to 6 carbon atoms, the divalent group represented by X² being selected from groups expressed by the following formulae:

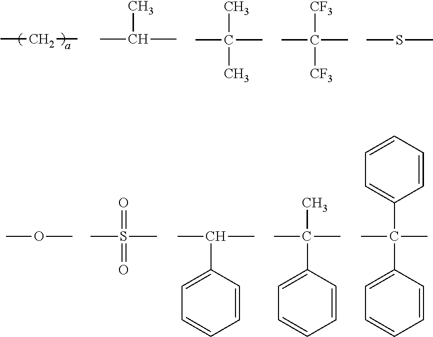

wherein a represents a number of 1 to 6.

[2]

The aromatic bismaleimide compound according to [1], wherein a number average molecular weight of the aromatic bismaleimide compound represented by the formula (1) is 3,000 to 50,000.

[3] The aromatic bismaleimide compound according to [1] or [2], wherein the divalent groups represented by X¹ in the formula (1) and X¹ in the formula (3) are identical to each other.
[4]

The aromatic bismaleimide compound according to any one of [1] to [3], wherein in the formula (1), when A¹ is represented by the formula (2), A² is represented by the formula (3); or when A¹ is represented by the formula (3), A² is represented by the formula (2).

[5]

A method for producing the aromatic bismaleimide compound according to any one of [1] to [4], comprising:

a step A of synthesizing an amic acid by reacting an aromatic diphthalic anhydride with an aromatic diamine at a molar ratio of aromatic diphthalic anhydride/aromatic diamine=1.01 to 1.50/1.0, and then performing cyclodehydration;

a step B subsequent to the step A, which is a step of synthesizing an amic acid with a reactant obtained in the step A and an aromatic diamine, and then performing cyclodehydration; and

a step C subsequent to the step B, which is a step of synthesizing a maleamic acid by reacting a reactant obtained in the step B with a maleic anhydride, and then performing cyclodehydration to block molecular chain ends with maleimide groups,

wherein the aromatic diphthalic anhydride used in the step A is represented by the following formula (4):

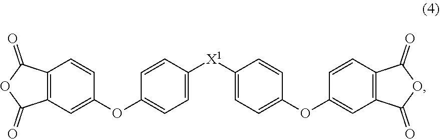

the aromatic diamine used in the step A is represented by the following formula (5):

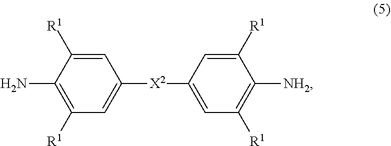

the aromatic diamine used in the step B is represented by the following formula (6):

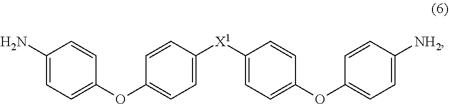

wherein in the formulae (4) and (6), X¹ independently represents a divalent group selected from groups expressed by the following formulae:

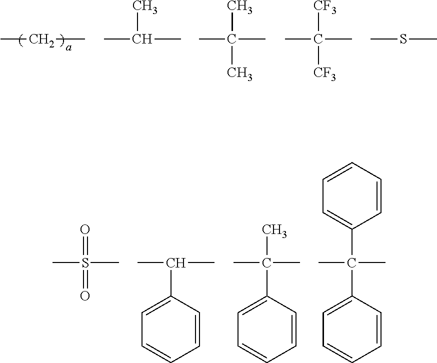

wherein a represents a number of 1 to 6; and

wherein in the formula (5), R¹ independently represents a hydrogen atom, a chlorine atom, or a substituted or unsubstituted aliphatic hydrocarbon group having 1 to 6 carbon atoms, X² independently represents a divalent group selected from groups expressed by the following formulae:

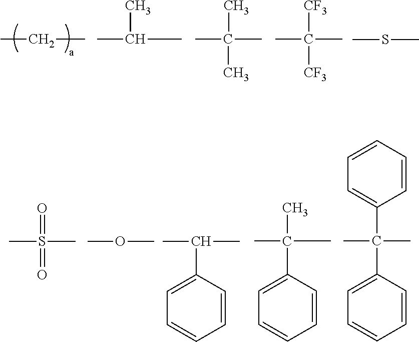

wherein a represents a number of 1 to 6.

[6]

A method for producing the aromatic bismaleimide compound according to any one of [1] to [4], comprising:

a step A′ of synthesizing an amic acid by reacting an aromatic diphthalic anhydride with an aromatic diamine at a molar ratio of aromatic diphthalic anhydride/aromatic diamine=1.01 to 1.50/1.0, and then performing cyclodehydration;

a step B′ subsequent to the step A′, which is a step of synthesizing an amic acid with a reactant obtained in the step A′ and an aromatic diamine, and then performing cyclodehydration; and

a step C′ subsequent to the step B′, which is a step of synthesizing a maleamic acid by reacting a reactant obtained in the step B′ with a maleic anhydride, and then performing cyclodehydration to block molecular chain ends with maleimide groups, wherein the aromatic diphthalic anhydride used in the step A′ is represented by the following formula (4):

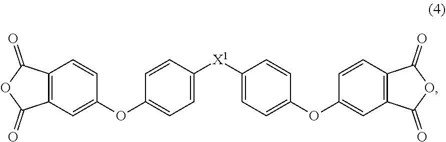

the aromatic diamine used in the step A′ is represented by the following formula (6):

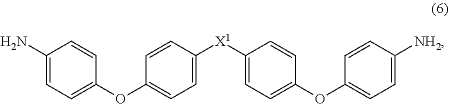

the aromatic diamine used in the step B′ is represented by the following formula (5):

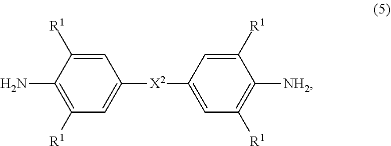

wherein in the formulae (4) and (6), X¹ independently represents a divalent group selected from groups expressed by the following formulae:

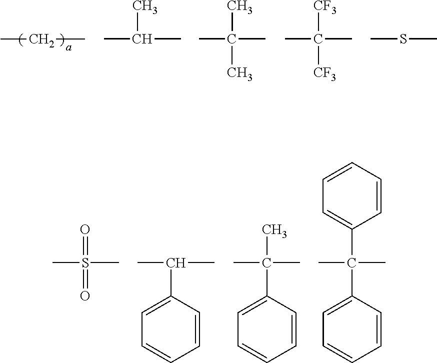

wherein a represents a number of 1 to 6; and

wherein in the formula (5), R¹ independently represents a hydrogen atom, a chlorine atom, or a substituted or unsubstituted aliphatic hydrocarbon group having 1 to 6 carbon atoms, X² independently represents a divalent group selected from groups expressed by the following formulae:

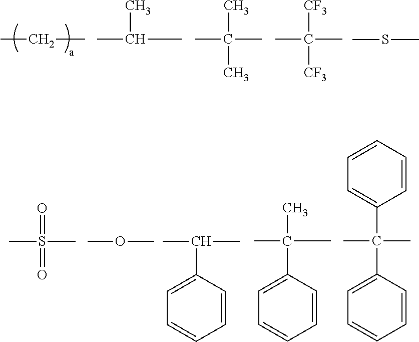

wherein a represents a number of 1 to 6.

[7]

A heat-curable cyclic imide resin composition comprising:

(A) the aromatic bismaleimide compound according to any one of [1] to [4];

(B) a reaction initiator; and

(C) an organic solvent.

[8]

The heat-curable cyclic imide resin composition according to [7], wherein the organic solvent (C) is at least one selected from the group consisting of methylethylketone (MEK), cyclohexanone, ethyl acetate, tetrahydrofuran (THF), isopropanol (IPA), xylene, toluene and anisole.

[9]

The heat-curable cyclic imide resin composition according to [7], wherein the reaction initiator (B) has a 1 hour half-life temperature of 80 to 115° C., and the composition is for use as a primer.

[10]

The heat-curable cyclic imide resin composition according to [9], wherein the organic solvent (C) is at least one selected from the group consisting of cyclohexanone, tetrahydrofuran (THF), isopropanol (IPA), xylene, toluene and anisole.

[11]

A method for producing a cured product, comprising:

curing the heat-curable cyclic imide resin composition according to [9] or [10] at a temperature of not higher than 150° C.

[12]

An adhesive agent composition, primer composition, composition for substrate or coating material composition comprising the heat-curable cyclic imide resin composition according to [7] or [8].

[13]

A cured product of the heat-curable cyclic imide resin composition according to [7] or [8].

[14]

A semiconductor device having the cured product of the heat-curable cyclic imide resin composition according to [13].

[15]

A substrate material having the cured product of the heat-curable cyclic imide resin composition according to [13].

The aromatic bismaleimide compound of the present invention can be turned into a film without using a film-forming agent, and is capable of being dissolved in a solvent other than a high-boiling aprotic polar solvent. The aromatic bismaleimide compound of the present invention with such property is useful as an adhesive agent, a primer, a coating material or the like.

Further, the heat-curable cyclic imide resin composition of the present invention can be cured at a low temperature without using an aprotic polar solvent such as NMP, and turned into a cured product superior in mechanical properties, heat resistance, relative permittivity, dielectric tangent, moisture resistance and adhesiveness. Thus, the heat-curable cyclic imide resin composition of the present invention is suitable for use in an adhesive agent, a substrate material, a primer and a coating material, and the cured product of this composition is suitable for use in a semiconductor device.

### BRIEF DESCRIPTION OF THE DRAWINGS

FIG. 1A is a ¹H-NMR spectrum chart of an aromatic bismaleimide compound synthesized in a working example 1.

FIG. 1B is a partially enlarged ¹H-NMR spectrum chart of the aromatic bismaleimide compound synthesized in the working example 1.

FIG. 2 is an IR spectrum chart of the aromatic bismaleimide compound synthesized in the working example 1.

### DETAILED DESCRIPTION OF THE INVENTION

Described hereunder are detailed embodiments of the present invention.

### Aromatic Bismaleimide Compound

The bismaleimide compound of the present invention is a novel aromatic bismaleimide compound represented by the following formula (1).

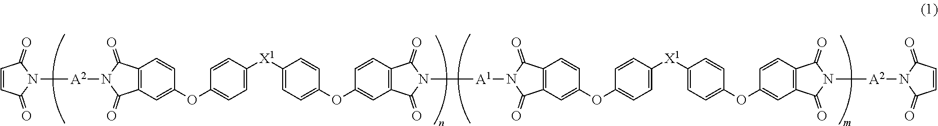

In the above formula (1), X¹ independently represents a divalent group selected from those expressed by the following formulae:

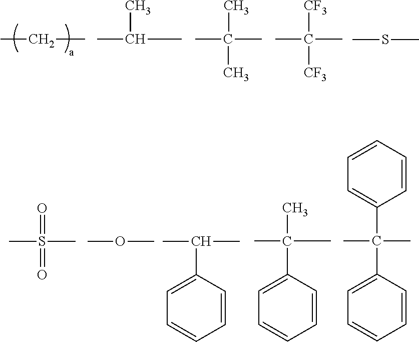

wherein a represents a number of 1 to 6.

In the above formula (1), m represents a number of 1 to 30, preferably a number of 2 to 20; n represents a number of 1 to 5, preferably a number of 1 to 3, and more preferably 1; each of A¹ and A² independently represents a divalent aromatic group expressed by the following formula (2) or (3).

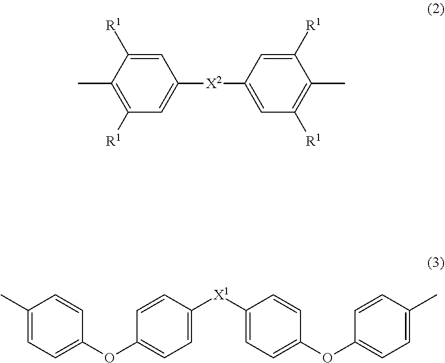

In the above formula (2), X² independently represents a divalent group selected from those expressed by the following formulae:

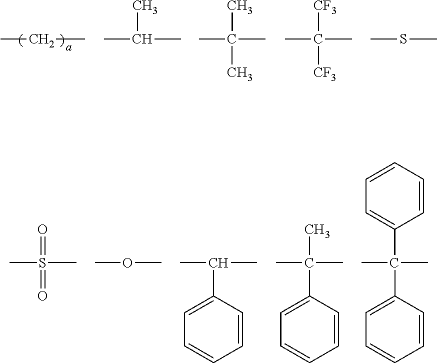

wherein a represents a number of 1 to 6.

In the above formula (2), R¹ independently represents a hydrogen atom, a chlorine atom, or a substituted or unsubstituted aliphatic hydrocarbon group having 1 to 6 carbon atoms. In the above formula (3), X¹ is defined as above.

In terms of raw material availability, —CH₂—, —C(CH₃)₂— are preferred as X¹. m represents a number of 1 to 30, preferably a number of 2 to 20. If m is in these ranges, there will be exhibited a favorable solubility of the above aromatic bismaleimide compound in a solution when the compound is in an uncured state; a favorable film-forming capability of the compound in the uncured state; and a favorable balance between the toughness and heat resistance of a cured product obtained. n represents a number of 1 to 5, preferably a number of 1 to 3, and more preferably 1.

In terms of raw material availability, —CH₂—, —C(CH₃)₂— are preferred as X². Further, R¹ independently represents a hydrogen atom, a chlorine atom, or a substituted or unsubstituted aliphatic hydrocarbon group having 1 to 6 carbon atoms. Examples of the substituted or unsubstituted aliphatic hydrocarbon group having 1 to 6 carbon atoms include a methyl group, an ethyl group, a n-propyl group, an isopropyl group, a n-butyl group, a t-butyl group and a cyclohexyl group; as well as groups obtained by substituting a part of or all the hydrogen atoms in any of these groups with halogen atoms such as F, Cl and Br, an example of which being a trifluoromethyl group. In terms of raw material availability, a hydrogen atom or a substituted or unsubstituted aliphatic hydrocarbon group having 1 to 3 carbon atoms are preferred as R¹; and it is more preferred that A¹ and A² differ from each other.

A number average molecular weight of the aromatic bismaleimide compound represented by the formula (1) is preferably 3,000 to 50,000, more preferably 5,000 to 40,000. When the number average molecular weight is in these ranges, the aromatic bismaleimide compound can be stably dissolved in a solvent such that a favorable film-forming capability will be exhibited as well.

Here, the number average molecular weight referred to in the present invention is a number average molecular weight measured by gel permeation chromatography (GPC) under the following conditions, using polystyrene as a reference material.

### [GPC Measurement Condition]

Developing solvent: tetrahydrofuran
Flow rate: 0.35 mL/min

### Detector: RI

Column: TSK-GEL H type (by TOSOH CORPORATION)
Column temperature: 40° C.
Sample injection volume: 5 μL

Further, the divalent groups represented by X¹ in the formula (1) and X¹ in the formula (3) are identical to each other. The aromatic bismaleimide compound of the present invention is produced by using a divalent acid anhydride and diamine each having an identical bisphenol backbone. The production method thereof is described in detail hereunder.

### Method for Producing Aromatic Bismaleimide Compound

There are no particular restrictions on a method for producing the aromatic bismaleimide compound of the present invention. The compound may, for example, be efficiently produced by any one of the methods shown below.

A first method for producing the aromatic bismaleimide compound includes a step A of synthesizing an amic acid with an aromatic diphthalic anhydride represented by the following formula (4) and an aromatic diamine represented by the following formula (5), and then performing cyclodehydration; a step B subsequent to the step A, which is a step of synthesizing an amic acid with the reactant obtained in the step A and an aromatic diamine represented by the following formula (6), and then performing cyclodehydration; and a step C subsequent to the step B, which is a step of synthesizing a maleamic acid by reacting the reactant obtained in the step B with a maleic anhydride, and then performing cyclodehydration to block molecular chain ends with maleimide groups.

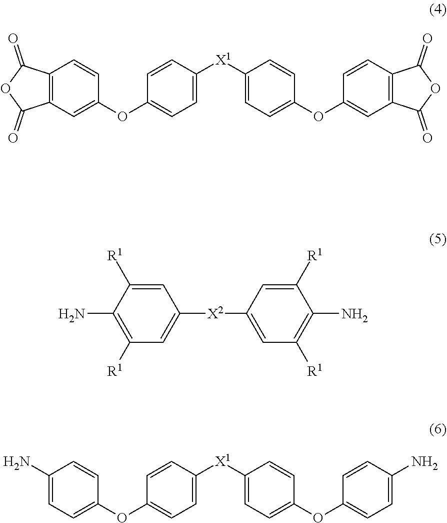

In the formula (4), X¹ is defined as above, and thus independently represents a divalent group selected from those expressed by the following formulae:

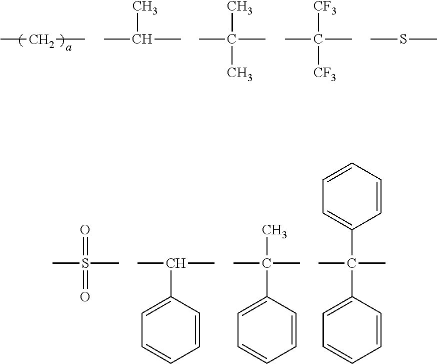

in which a represents a number of 1 to 6.

In the formula (5), R¹ and X² are defined as above i.e. R¹ independently represents a hydrogen atom, a chlorine atom, or a substituted or unsubstituted aliphatic hydrocarbon group having 1 to 6 carbon atoms, and X² independently represents a divalent group selected from those expressed by the following formulae:

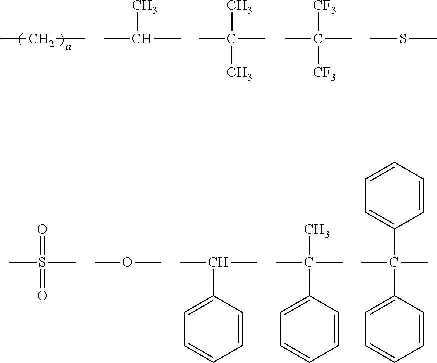

in which a represents a number of 1 to 6.

In the formula (6), X¹ is defined as above.

A second method for producing the aromatic bismaleimide compound includes a step A′ of synthesizing an amic acid with an aromatic diphthalic anhydride represented by the following formula (4) and an aromatic diamine represented by the following formula (6), and then performing cyclodehydration; a step B′ subsequent to the step A′, which is a step of synthesizing an amic acid with the reactant obtained in the step A′ and an aromatic diamine represented by the following formula (5), and then performing cyclodehydration; and a step C′ subsequent to the step B′, which is a step of synthesizing a maleamic acid by reacting the reactant obtained in the step B′ with a maleic anhydride, and then performing cyclodehydration to block molecular chain ends with maleimide groups.

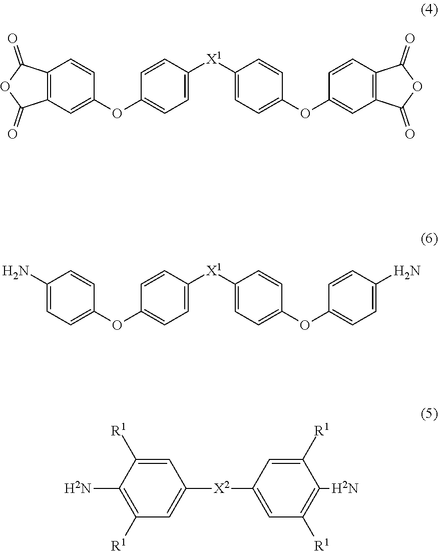

In the formula (4), X¹ is defined as above, and thus independently represents a divalent group selected from those expressed by the following formulae:

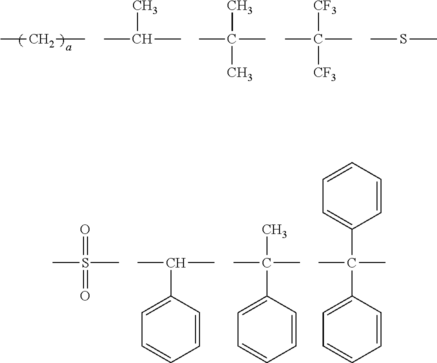

in which a represents a number of 1 to 6.

In the formula (6), X¹ is defined as above.

In the formula (5), R¹ and X² are defined as above i.e. R¹ independently represents a hydrogen atom, a chlorine atom, or a substituted or unsubstituted aliphatic hydrocarbon group having 1 to 6 carbon atoms, and X² independently represents a divalent group selected from those expressed by the following formulae:

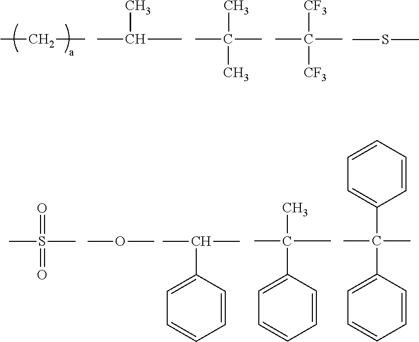

in which a represents a number of 1 to 6.

The two production methods have now been described. As a basic pattern, the aromatic bismaleimide compound can be obtained by the step A (or step A′) of synthesizing an amic acid with an aromatic diphthalic anhydride and an aromatic diamine, and then performing cyclodehydration; the step B (or step B′) subsequent to the step A (or step A′), which is a step of synthesizing an amic acid by adding an aromatic diamine other than that employed in the previous step A (or step A′), and then further performing cyclodehydration; and then the step C (or step C′) subsequent to the step B (or step B′), which is a step of reacting a maleic anhydride to synthesize a maleamic acid, and then finally performing cyclodehydration to block molecular chain ends with maleimide groups. The above two production methods mainly differ from each other only in the order in which the different types of aromatic diamines are added.

The reactions can be grouped into two categories which are the synthesis reaction of an amic acid or maleamic acid; and the cyclodehydration reaction. These reactions are described in detail hereunder.

In the step A (or step A′), an amic acid is at first synthesized by reacting a particular aromatic diphthalic anhydride with a particular aromatic diamine. This reaction usually proceeds in a high-boiling aprotic polar solvent and at a temperature of room temperature (25° C.) to 100° C. However, in the reaction of an aromatic diphthalic anhydride and an aromatic diamine, anisole and a derivative(s) thereof (e.g. o-methylanisole, p-methylanisole) may be used as a solvent instead of a high-boiling aprotic polar solvent.

Next, the cyclodehydration reaction of the amic acid is performed in a way such that after reacting the amic acid at a temperature of 120 to 180° C., the cyclodehydration reaction is then caused to proceed while removing from the system a water produced as a by-product due to a condensation reaction. A high-boiling aprotic polar solvent and/or an acid catalyst may also be added to promote the cyclodehydration reaction. Examples of the high-boiling aprotic polar solvent include N,N-dimethylformamide (DMF) and dimethylsulfoxide (DMSO). Any one of these high-boiling aprotic polar solvents may be used alone, or two or more of them may be used in combination. Further, examples of the acid catalyst include sulfuric acid, methanesulfonic acid and trifluoromethanesulfonic acid. Any one of these acid catalysts may be used alone, or two or more of them may be used in combination.

A compound ratio between the aromatic diphthalic anhydride and the aromatic diamine is preferably aromatic diphthalic anhydride/aromatic diamine=1.01 to 1.50/1.0, more preferably aromatic diphthalic anhydride/aromatic diamine=1.01 to 1.15/1.0, in terms of molar ratio. By combining the aromatic diphthalic anhydride and the aromatic diamine at this ratio, there can be synthesized, as a result, a copolymer having an imide group at both ends.

In the step B (or step B′), an amic acid is at first synthesized by reacting the copolymer obtained in the step A (or step A′) with a particular aromatic diamine, the copolymer being that having an imide group at both ends. This reaction usually proceeds in a high-boiling aprotic polar solvent and at a temperature of room temperature (25° C.) to 100° C. However, in the reaction of the aromatic diamine and the copolymer having an imide group at both ends, anisole and a derivative(s) thereof (e.g. o-methylanisole, p-methylanisole) may be preferably used as a solvent instead of a high-boiling aprotic polar solvent. Any one of such anisole and the derivatives thereof may be used alone, or two or more of them may be used in combination.

Likewise, the subsequent cyclodehydration reaction of the amic acid is performed in a way such that after reacting the amic acid at a temperature of 120 to 180° C., the cyclodehydration reaction is then caused to proceed while removing from the system a water produced as a by-product due to a condensation reaction. A high-boiling aprotic polar solvent and/or an acid catalyst may also be added to promote the cyclodehydration reaction. Examples of the high-boiling aprotic polar solvent include N,N-dimethylformamide (DMF) and dimethylsulfoxide (DMSO). Any one of these high-boiling aprotic polar solvents may be used alone, or two or more of them may be used in combination. Further, examples of the acid catalyst include sulfuric acid, methanesulfonic acid and trifluoromethanesulfonic acid. Any one of these acid catalysts may be used alone, or two or more of them may be used in combination.

A compound ratio between the copolymer having an imide group at both ends and the aromatic diamine is preferably 1.0:1.6 to 2.5, more preferably 1.0:1.8 to 2.2, in terms of molar ratio.

In the step C (or step C′), a maleamic acid is synthesized by reacting, at a temperature of room temperature (25° C.) to 100° C., a diamine having an amino group at both ends with a maleic anhydride, the diamine being that obtained in the step B (or B′). Finally, cyclodehydration is performed while removing from the system a water produced at 120 to 180° C. as a by-product, thereby blocking the molecular chain ends with maleimide groups, thus obtaining the target aromatic bismaleimide compound.

A compound ratio between the diamine having an amino group at both ends and the maleic anhydride is preferably 1.0:1.6 to 2.5, more preferably 1.0:1.8 to 2.2, in terms of molar ratio.

### Heat-Curable Cyclic Imide Resin Composition

The heat-curable cyclic imide resin composition of the present invention contains (A) the abovementioned aromatic bismaleimide compound, (B) a reaction initiator and (C) an organic solvent.

### (A) Aromatic Bismaleimide Compound

One kind of such aromatic bismaleimide compound as the component (A) may be used alone, or two or more kinds thereof may be used in combination.

It is preferred that the component (A) be contained in the composition of the present invention by an amount of 2.5 to 50% by mass, more preferably 4 to 45% by mass, and even more preferably 5 to 40% by mass.

### (B) Reaction Initiator

A reaction initiator as a component (B) is added to promote a cross-linking reaction of the aromatic bismaleimide as the component (A). There are no particular restrictions on the component (B) so long as it is capable of promoting the cross-linking reaction. Examples of the component (B) include ion catalysts such as imidazoles, tertiary amines, quaternary ammonium salts, borontrifluoride-amine complexes, organo-phosphines and organo-phosphonium salts; and radical polymerization initiators such as organic peroxides, hydroperoxide and azo-iso-butyronitrile. Among these reaction initiators, imidazoles and organic peroxides are preferred. Examples of the imidazoles include 2-methylimidazole, 2-ethyl-4-methylimidazole, 2-phenylimidazole, 1-benzyl-2-phenylimidazole and 2-phenyl-4,5-dihydroxymethylimidazole. Examples of the organic peroxides include dicumylperoxide, t-butylperoxy benzoate, t-amylperoxy benzoate, dibenzoyl peroxide, dilauroyl peroxide, 2-ethylhexanoic acid-t-amyl peroxide and 1,6-bis(tert-butylperoxycarbonyloxy)hexane.

When the composition of the present invention is used as a primer for a copper substrate, it is preferred that the reaction initiator as the component (B) be a reaction initiator (organic peroxide) having a 1 hour half-life temperature of 80 to 115° C. Examples of such reaction initiator (organic peroxide) having a 1 hour half-life temperature of 80 to 115° C. include the following compounds (temperatures in the brackets are the 1 hour half-life temperatures of the compounds.)

Dibenzoyl peroxide (92.0° C.)
2-ethylhexanoic acid-t-amyl peroxide (88.0° C.)
1,6-bis(tert-butylperoxycarbonyloxy)hexane (115.0° C.)

One kind of such reaction initiator as the component (B) may be used alone, or two or more kinds thereof may be used in combination.

It is preferred that the reaction initiator be added in an amount of 0.05 to 10 parts by mass, more preferably 0.1 to 5 parts by mass, per 100 parts by mass of the component (A). When the amount of the reaction initiator added is out of these ranges, the cured product may exhibit a poor balance between heat resistance and moisture resistance, and a curing speed may be either extremely slow or extremely fast at the time of performing molding.

### (C) Organic Solvent

The composition of the present invention further contains an organic solvent as a component (C). There are no particular restrictions on the kind(s) of such organic solvent so long as it is capable of dissolving the component (A). Here, the expression “the component (C) is capable of dissolving the component (A)” refers to a state where after adding 25% by mass of the component (A) to the component (C), one cannot visually recognize any undissolved component (A) at 25° C.

Examples of the component (C) include general organic solvents such as methylethylketone (MEK), cyclohexanone, ethyl acetate, tetrahydrofuran (THF), isopropanol (IPA), xylene, toluene and anisole. Any one of these organic solvents may be used alone, or two or more of them may be used in combination.

When the composition of the present invention is used as a primer for a copper substrate, it is preferred that the organic solvent as the component (C) be, for example, cyclohexanone, tetrahydrofuran (THF), isopropanol (IPA), xylene, toluene and/or anisole.

In terms of the solubility of the component (A), it is preferred that organic solvents such as anisole, xylene and toluene be used. Meanwhile, it is preferred that aprotic polar solvents such as dimethylsulfoxide (DMSO), dimethylformamide (DMF) and N-methyl-2-pyrolidone (NMP) be not used due to the fact that they have high boiling points and are toxic. Unlike a conventional composition containing a polyimide compound capable of being dissolved only in an aprotic polar solvent, the composition of the present invention has the advantage that there is no need to use these aprotic polar solvents.

### Other Additives

Various additives may be added to the heat-curable cyclic imide resin composition of the present invention, provided that the effects of the invention are not impaired. For example, in order to improve resin properties, there may be added a heat-curable resin such as an acrylic resin and an epoxy resin; an organopolysiloxane; a silicone oil; a thermoplastic resin; a thermoplastic elastomer; an organic synthetic rubber; a light stabilizer; a polymerization inhibitor; a flame retardant; a pigment; a dye; and an adhesion aid. Further, in order to improve electric properties, there may be added, for example, an ion trapping agent. Furthermore, in order to improve dielectric properties, there may be added, for example, a fluorine-containing material. An inorganic filler such as silica may also be added for the purpose of adjusting a coefficient of thermal expansion (CTE).

The heat-curable cyclic imide resin composition of the present invention can be used as an adhesive agent, a primer, a coating material for semiconductor devices, and a material for substrates. There are no particular restrictions on a method and form by which the composition of the invention is used.

Usage examples are shown below; the present invention shall not be limited to these examples.

For example, the heat-curable cyclic imide resin composition containing the components (A), (B) and (C) is to be applied to a base material, followed by heating this base material at a temperature of usually not lower than 80° C., preferably not lower than 100° C. for 0.5 to 5 hours so as to eliminate the organic solvent. By further heating the base material at a temperature of not lower than 150° C., preferably not lower than 175° C. for 0.5 to 10 hours, there can be formed a strong cyclic imide coating film with a flat surface. In order to efficiently eliminate the organic solvent in the composition and allow the reaction of the resin to progress effectively, the curing temperature may be raised in a stepwise manner in certain cases. The cured product (coating film) obtained by curing the composition of the present invention is superior in mechanical properties, heat resistance, relative permittivity, dielectric tangent, moisture resistance and adhesiveness. Thus, the cured product of the present invention can be utilized as, for example, a passivation film formed on semiconductor element surfaces; a junction protective film for protecting junctional regions between diodes, transistors or the like; an α-ray shielding film for VLSI; an interlayer insulation film; an ion implantation mask; a conformal coating film for printed circuit boards; an orientation film for liquid crystal display elements; a protective film for glass fibers; and a surface protection film for solar cells.

As a method for applying the composition of the invention, methods using a spin coater, a slit coater, a sprayer, a dip coater, a bar coater or the like may be used. However, there are no particular restrictions on such method.

After forming the cured product (coating film), by molding an epoxy resin molding material for semiconductor encapsulation onto this cured product (coating film), an adhesion between the epoxy resin molding material for semiconductor encapsulation and the base material can be improved. A semiconductor device thus obtained has a high reliability as the epoxy resin molding material for semiconductor encapsulation exhibits no cracks and no peeling from the base material in a solder reflow step following moisture absorption.

In this case, as an epoxy resin molding material for semiconductor encapsulation, there may be used a known epoxy resin composition for semiconductor encapsulation that contains, for example, an epoxy resin having at least two epoxy groups in one molecule; a phenolic resin; a curing agent for an epoxy resin, such as acid anhydride; and/or an inorganic filler. A commercially available composition of such kind may also be used.

In a case where an easily-oxidizable metal such as copper is used as the base material, it is preferred that an environment for curing the heat-curable cyclic imide resin composition and the epoxy resin molding material for semiconductor encapsulation be a nitrogen atmosphere for the sake of oxidation prevention.

The composition of the present invention may also be applied to a sheet base material, and then used in the form of a film. As such sheet base material, those that are generally used may be used, examples of which include polyolefin resins such as polyethylene (PE) resin, polypropylene (PP) resin and polystyrene (PS) resin; and polyester resins such as polyethylene terephthalate (PET) resin, polybutylene terephthalate (PBT) resin and polycarbonate (PC) resin. The surfaces of these resins may be subjected to a release treatment.

Further, there are no particular restrictions on a method for applying the composition of the present invention, examples of which include methods using a gap coater, a curtain coater, a roll coater, a laminator or the like. There are also no particular restrictions on a thickness of a coating layer. However, it is preferred that the thickness of the coating layer be 1 to 100 μm, more preferably 3 to 80 μm, after distilling away the solvent.

Here, a cover film may also be provided on the coating layer. Moreover, a copper foil may be attached to the coating layer such that the substrate material may then be used as a resin-attached copper foil.

One embodiment of the composition of the present invention is a primer composition for copper as a base material. In the case of a primer composition for copper as a base material, by using, as the component (B), an organic peroxide having the 1 hour half-life temperature of 80 to 115° C., the primer composition will be able to be cured at a low temperature, and the copper substrate can thus be restricted from being oxidized and discolored accordingly even when curing has taken place under an air atmosphere. If used as a primer composition for a copper base material, it is preferred that the heat-curable cyclic imide resin composition be cured at a temperature of not higher than 150° C. under an air atmosphere; this is preferable because there is no need to purposely prepare, for example, a device enabling curing even under a nitrogen atmosphere. Here, it is not preferable to perform curing reaction under an atmosphere where oxygen is present at a high concentration, such as an oxygen atmosphere, because there are concerns that an adhesion durability will deteriorate, and that a volatilized solvent will catch fire easily. As described above, provided that the curing temperature is not higher than 150° C., the heat-curable cyclic imide resin composition may be applied to the copper base material, followed by heating this base material at a temperature i.e. first curing temperature of usually not lower than 80° C., preferably not lower than 100° C. for 0.5 to 5 hours so as to eliminate the organic solvent, and then by further heating this base material at a temperature i.e. second curing temperature of not higher than 150° C. for 0.5 to 10 hours, the second curing temperature being higher than the first curing temperature accordingly.

### WORKING EXAMPLE

The present invention is described in detail hereunder with reference to working and comparative examples. However, the invention shall not be limited to the following working examples. Here, in the working and comparative examples, the term “room temperature” refers to 25° C.

A number average molecular weight (Mn) mentioned hereunder is measured by gel permeation chromatography (GPC) under the following measurement conditions, using polystyrene as a reference material.

[GPC measurement condition]
Developing solvent: tetrahydrofuran
Flow rate: 0.35 mL/min

### Detector: RI

Column: TSK-GEL H type (by TOSOH CORPORATION)
Column temperature: 40° C.
Sample injection volume: 5 μL

### Working Example 1

### Production of Bismaleimide Compound

An amic acid was synthesized by adding 65.06 g (0.125 mol) of 2,2-bis[4-(2,3-dicarboxyphenoxy)phenyl]propane dianhydride, 35.26 g (0.115 mol) of 4,4-methylenebis(2,6-diethylaniline) and 250 g of anisole to a 1 L glass four-necked flask equipped with a stirrer, a Dean-Stark apparatus, a cooling condenser and a thermometer, and then stirring them at 80° C. for three hours. Later, the temperature was directly raised to 150° C., and stirring was performed for another two hours while distilling away a water produced as a by-product, thereby synthesizing a copolymer.

Next, an amic acid was synthesized by adding 7.05 g (0.015 mol) of 2,2-bis[4-(4-aminophenoxy)phenyl]propane to the flask containing the copolymer solution that had been cooled to room temperature, and then performing stirring at 80° C. for three hours. Later, the temperature was directly raised to 150° C., and stirring was performed for another two hours while distilling away a water produced as a by-product, thereby synthesizing a diamine compound with each end being blocked with an amino group.

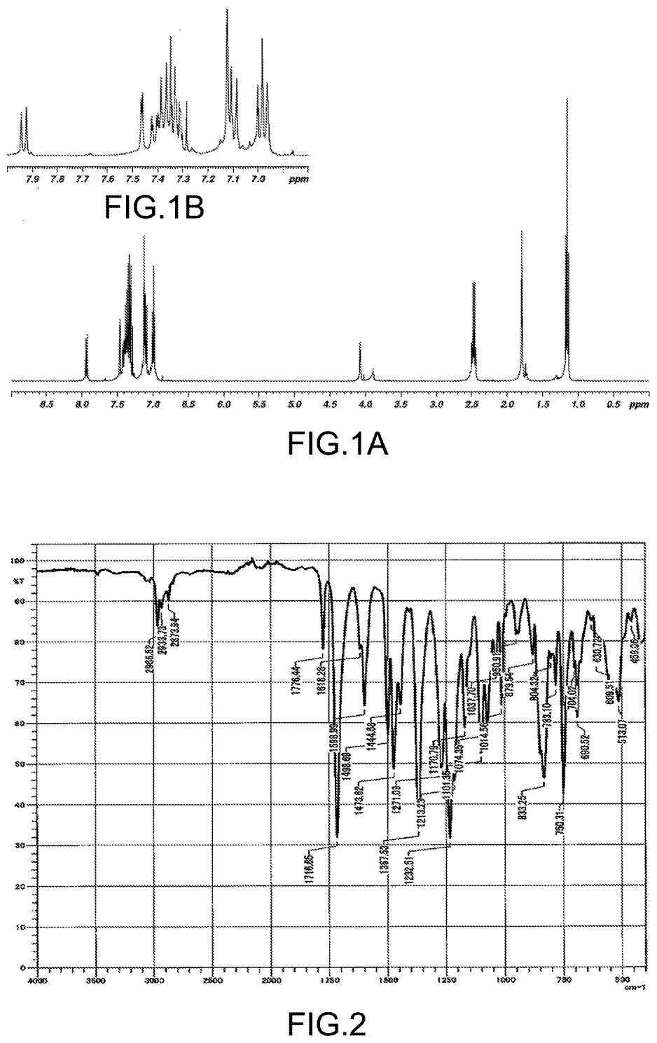

A maleamic acid was synthesized by adding 1.45 g (0.015 mol) of a maleic anhydride to the flask that had been cooled to room temperature and now contained the solution of the obtained diamine compound, and then performing stirring at 80° C. for three hours. Later, the temperature was directly raised to 150° C., and stirring was performed for another two hours while distilling away a water produced as a by-product, thereby obtaining a varnish of an aromatic bismaleimide compound as a target substance. Next, anisole was distilled away at 130° C. under a reduced pressure (10 mmHg or lower) to obtain a dark brown solid. A ¹H-NMR and IR spectra of the product obtained indicate that this product has a structure represented by the following formula (A-1). The ¹H-NMR spectrum is shown in FIGS. 1A and 1B, and the IR spectrum is shown in FIG. 2. FIG. 1B is diagram obtained by partially enlarging the ¹H-NMR spectrum shown in FIG. 1A. Further, a number average molecular weight of the product obtained was 11,600.

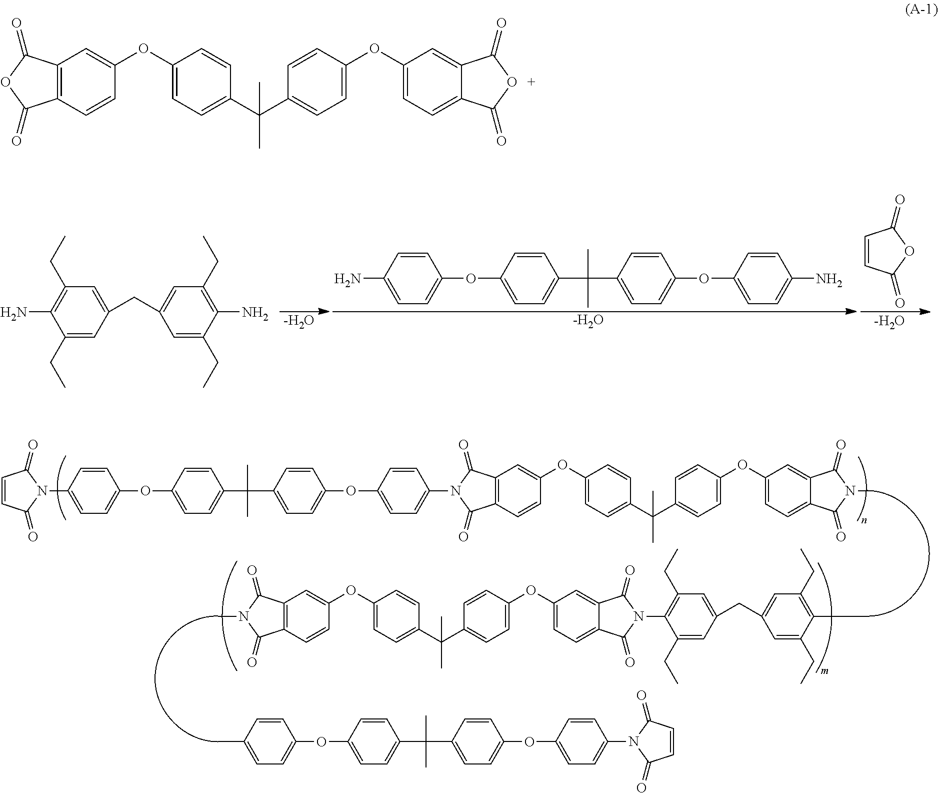

m=8, n=1 (both are average values)

¹H-NMR (400 MHz, CDCl₃) δ1.26-1.28 (—C₆H₂(—CH₂—CH₃)₂, 12H), 1.72-1.78 (—C(CH₃)₂—, 12H), 2.45-2.52 (—C₆H₂ (—CH₂—CH₃)₂, 8H), 3.7 (—C₆H₂—CH₂—C₆H₂—, 2H), 4.14 (—CH═CH—, 4H), 6.65-7.05 (derived from aromatic ring, 14H), 7.06-7.14 (derived from aromatic ring, 8H), 7.28-7.48 (derived from aromatic ring, 24H), 7.92-7.95 (derived from aromatic ring, 2H)

### Working Example 2

### Production of Bismaleimide Compound

An amic acid was synthesized by adding 65.06 g (0.125 mol) of 2,2-bis[4-(2,3-dicarboxyphenoxy)phenyl]propane dianhydride, 54.05 g (0.115 mol) of 2,2-bis[4-(4-aminophenoxy)phenyl]propane and 250 g of anisole to a 1 L glass four-necked flask equipped with a stirrer, a Dean-Stark apparatus, a cooling condenser and a thermometer, and then stirring them at 80° C. for six hours. Later, the temperature was directly raised to 150° C., and stirring was performed for another two hours while distilling away a water produced as a by-product, thereby synthesizing a copolymer.

Next, an amic acid was synthesized by adding 4.60 g (0.015 mol) of 4,4-methylenebis(2,6-diethylaniline) to the flask containing the copolymer solution that had been cooled to room temperature, and then performing stirring at 80° C. for three hours. Later, the temperature was directly raised to 150° C., and stirring was performed for another two hours while distilling away a water produced as a by-product, thereby synthesizing a diamine compound with each end being blocked with an amino group.

A maleamic acid was synthesized by adding 1.45 g (0.015 mol) of a maleic anhydride to the flask that had been cooled to room temperature and now contained the solution of the obtained diamine compound, and then performing stirring at 80° C. for three hours. Later, the temperature was directly raised to 150° C., and stirring was performed for another two hours while distilling away a water produced as a by-product, thereby obtaining a varnish of an aromatic bismaleimide compound as a target substance. Next, anisole was distilled away at 130° C. under a reduced pressure (10 mmHg or lower) to obtain a dark brown solid having a structure represented by the following formula (A-2). Further, a number average molecular weight of the product obtained was 15,100.

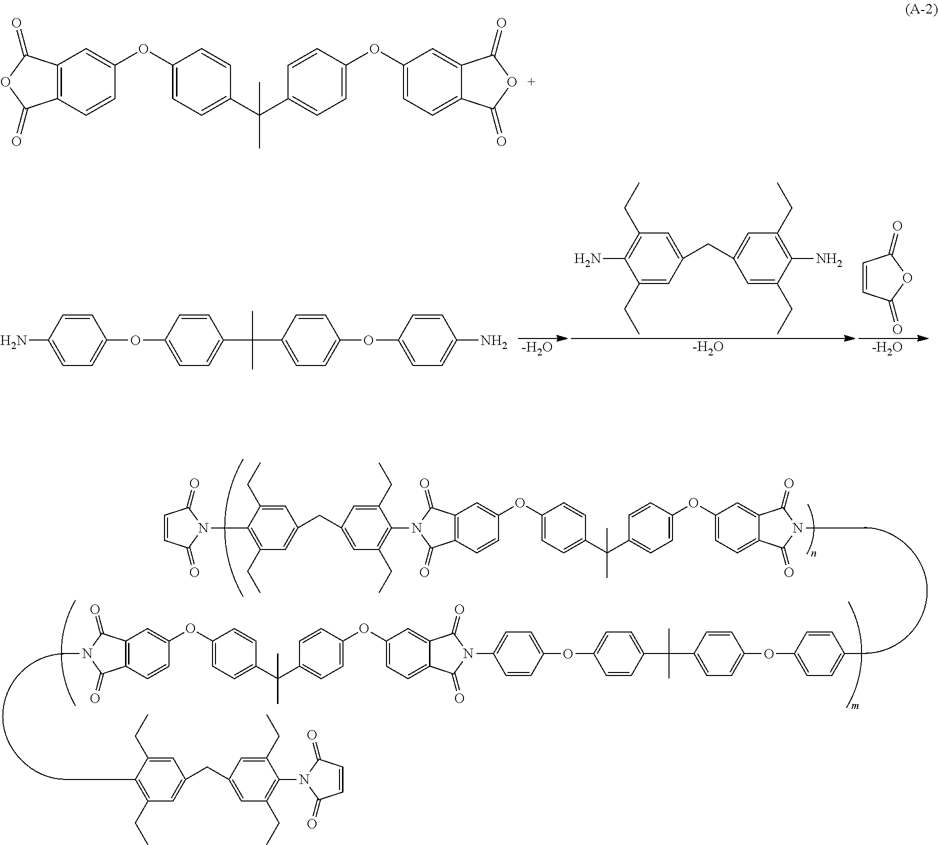

m=8, n=1 (both are average values)

### Working Example 3

### Production of Bismaleimide Compound

An amic acid was synthesized by adding 65.06 g (0.125 mol) of 2,2-bis[4-(2,3-dicarboxyphenoxy)phenyl]propane dianhydride, 40.78 g (0.115 mol) of 4,4-methylenebis(2,6-dipropylaniline) and 250 g of anisole to a 1 L glass four-necked flask equipped with a stirrer, a Dean-Stark apparatus, a cooling condenser and a thermometer, and then stirring them at 80° C. for three hours. Later, the temperature was directly raised to 150° C., and stirring was performed for another two hours while distilling away a water produced as a by-product, thereby synthesizing a copolymer.

Next, an amic acid was synthesized by adding 7.05 g (0.015 mol) of 2,2-bis[4-(4-aminophenoxy)phenyl]propane to the flask containing the copolymer solution that had been cooled to room temperature, and then performing stirring at 80° C. for three hours. Later, the temperature was directly raised to 150° C., and stirring was performed for another two hours while distilling away a water generated, thereby synthesizing a diamine compound with each end being blocked with an amino group.

A maleamic acid was synthesized by adding 1.45 g (0.015 mol) of a maleic anhydride to the flask that had been cooled to room temperature and now contained the solution of the obtained diamine compound, and then performing stirring at 80° C. for three hours. Later, the temperature was directly raised to 150° C., and stirring was performed for another two hours while distilling away a water produced as a by-product, thereby obtaining a varnish of an aromatic bismaleimide compound as a target substance. Next, anisole was distilled away at 130° C. under a reduced pressure (10 mmHg or lower) to obtain a dark brown solid having a structure represented by the following formula (A-3). Further, a number average molecular weight of the product obtained was 12,500.

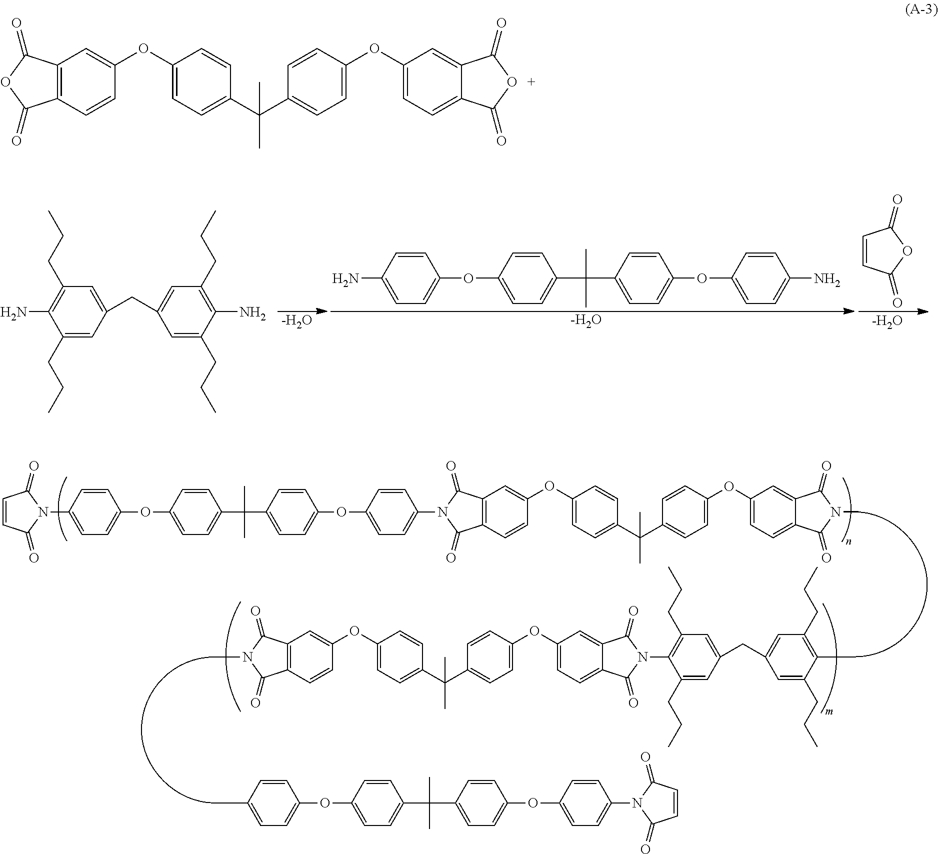

m=8, n=1 (both are average values)

With regard to the bismaleimide compounds obtained (working examples 1 to 3) and the following bismaleimide compounds (comparative examples 1 to 3), the methods shown below were used to study the solubility of each compound in various organic solvents as well as a film-forming capability thereof. The results are shown in Table 1.

### Comparative Example 1: 4,4′-diphenylmethanebismaleimide (by K.I Chemical Industry Co., LTD.)

### Comparative Example 2: 2,2′-bis-[4-(4-maleimidephenoxy)phenyl]propane (by K.I Chemical Industry Co., LTD.)

### Comparative Example 3: Long-Chain Alkyl Group-Containing Bismaleimide Compound (BMI-1500 by Designer Molecules Inc.)

### Solubility Test

Each bismaleimide compound was dissolved in 100 g of an organic solvent (anisole, tetrahydrofuran (THF), N-methyl-2-pyrolidone (NMP) or N,N-dimethylformamide (DMF)) at 25° C., followed by measuring a dissolved amount (g/100 g solvent).

### Method for Evaluating Film-Forming Capability

Using a Baker type applicator, a N,N-dimethylformamide (DMF) solution of each bismaleimide compound (active ingredient 50% by mass) was applied to a polyethylene terephthalate (PET) film (G2-38 by TEIJIN LIMITED.) having a thickness of 38 μm in a way such that the solution applied thereto would have a thickness of 30 μm and a size of A4 (210 mm×297 mm). The solution applied was then dried at 150° C. After drying, “∘” was given to examples where the solution had been turned into a film without any difficulty, and a clean appearance was thus observed; whereas “x” was given to examples where, for example, the solution had failed to be turned into a film due to crawling, or a poor appearance was observed as agglomeration had occurred due to a precipitation of bismaleimide.

TABLE 1

| | Solubility test (g/100 g Solvent) | | | | Film-forming |
|---|---|---|---|---|---|
| | Anisole | THF | NMP | DMF | capability |
| Working | >80 | >30 | >100 | >150 | ∘ |
| example 1 | | | | | |
| Working | >80 | >35 | >100 | >150 | ∘ |
| example 2 | | | | | |
| Working | >80 | >30 | >90 | >150 | ∘ |
| example 3 | | | | | |
| Comparative | <5 | <10 | <80 | >100 | x |
| example 1 | | | | | |
| Comparative | <5 | <30 | <80 | >100 | x |
| example 2 | | | | | |
| Comparative | >80 | >30 | >100 | >120 | x |
| example 3 | | | | | |

### Working Example 4

An amic acid was synthesized by adding 65.06 g (0.125 mol) of 2,2-bis[4-(2,3-dicarboxyphenoxy)phenyl]propane dianhydride, 35.26 g (0.115 mol) of 4,4-methylenebis(2,6-diethylaniline) and 250 g of anisole to a 1 L glass four-necked flask equipped with a stirrer, a Dean-Stark apparatus, a cooling condenser and a thermometer, and then stirring them at 80° C. for three hours. Later, the temperature was directly raised to 150° C., and stirring was performed for another two hours while distilling away a water produced as a by-product, thereby synthesizing a copolymer.

Next, an amic acid was synthesized by adding 7.05 g (0.015 mol) of 2,2-bis[4-(4-aminophenoxy)phenyl]propane to the flask containing the copolymer solution that had been cooled to room temperature, and then performing stirring at 80° C. for three hours. Later, the temperature was directly raised to 150° C., and stirring was performed for another two hours while distilling away a water produced as a by-product, thereby synthesizing a diamine compound with each end being blocked with an amino group.

A maleamic acid was synthesized by adding 1.45 g (0.015 mol) of a maleic anhydride to the flask that had been cooled to room temperature and now contained the solution of the obtained diamine compound, and then performing stirring at 80° C. for three hours. Later, the temperature was directly raised to 150° C., and stirring was performed for another two hours while distilling away a water produced as a by-product, thereby obtaining a varnish of an aromatic bismaleimide compound represented by the following formula (A-1). The number average molecular weight (Mn) of this aromatic bismaleimide compound was 11,500. Anisole was then added to the varnish in a way such that non-volatile constituents would be in an amount of 16% by mass, followed by adding 2 parts by mass of dicumylperoxide per 100 parts by mass of the non-volatile constituents, and then keeping performing stirring under room temperature until the dicumylperoxide had dissolved, thereby obtaining a composition.

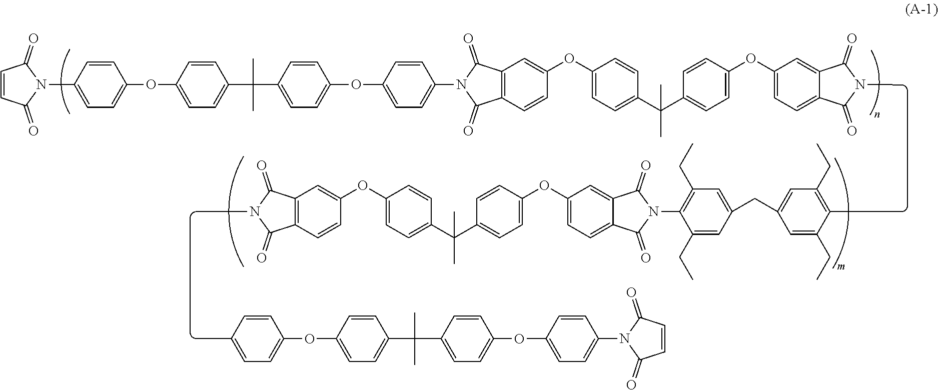

m=8, n=1 (both are average values)

### Working Example 5

An amic acid was synthesized by adding 65.06 g (0.125 mol) of 2,2-bis[4-(2,3-dicarboxyphenoxy)phenyl]propane dianhydride, 40.78 g (0.115 mol) of 4,4-methylenebis(2,6-dipropylaniline) and 250 g of anisole to a 1 L glass four-necked flask equipped with a stirrer, a Dean-Stark apparatus, a cooling condenser and a thermometer, and then stirring them at 80° C. for three hours. Later, the temperature was directly raised to 150° C., and stirring was performed for another two hours while distilling away a water produced as a by-product, thereby synthesizing a copolymer.

Next, an amic acid was synthesized by adding 7.05 g (0.015 mol) of 2,2-bis[4-(4-aminophenoxy)phenyl]propane to the flask containing the copolymer solution that had been cooled to room temperature, and then performing stirring at 80° C. for three hours. Later, the temperature was directly raised to 150° C., and stirring was performed for another two hours while distilling away a water generated, thereby synthesizing a diamine compound with each end being blocked with an amino group.

A maleamic acid was synthesized by adding 1.45 g (0.015 mol) of a maleic anhydride to the flask that had been cooled to room temperature and now contained the solution of the obtained diamine compound, and then performing stirring at 80° C. for three hours. Later, the temperature was directly raised to 150° C., and stirring was performed for another two hours while distilling away a water produced as a by-product, thereby obtaining a varnish of an aromatic bismaleimide compound represented by the following formula (A-3). The number average molecular weight (Mn) of this aromatic bismaleimide compound was 12,500. Anisole was then added to the varnish in a way such that non-volatile constituents would be in an amount of 16% by mass, followed by adding 2 parts by mass of dicumylperoxide per 100 parts by mass of the non-volatile constituents, and then keeping performing stirring under room temperature until the dicumylperoxide had dissolved, thereby obtaining a composition.

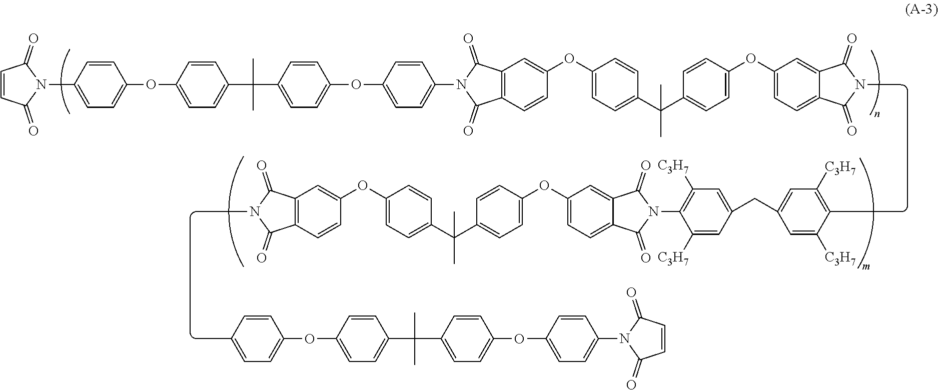

m=8, n=1 (both are average values)

### Working Example 6

Synthesis was performed in a similar manner as the working example 4, except that the amount of 4,4-methylenebis(2,6-diethylaniline) added in the working example 4 was now changed from 35.26 g (0.115 mol) to 61.32 g (0.220 mol). As a result, an aromatic bismaleimide compound represented by the following formula (A-4) was obtained. The number average molecular weight (Mn) of the aromatic bismaleimide compound obtained was 3,500. A varnish was also prepared in a similar manner as the working example 4 after synthesis.

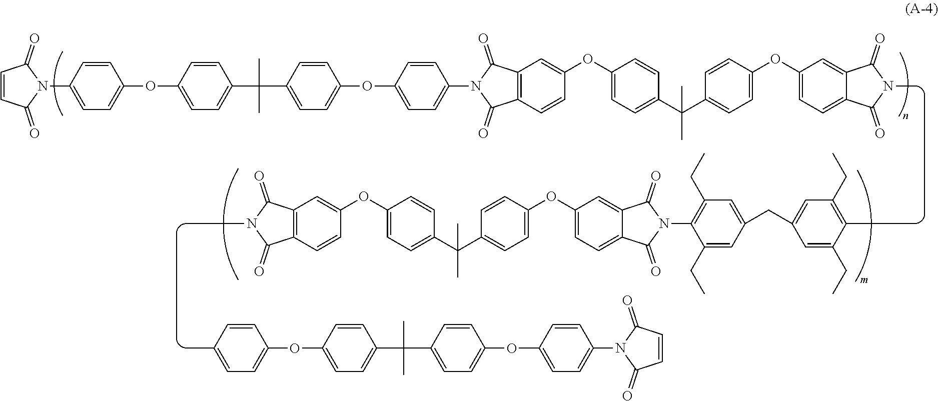

m=1, n=1 (both are average values)

### Working Example 7

Synthesis was performed in a similar manner as the working example 4, except that the amount of 4,4-methylenebis(2,6-diethylaniline) added in the working example 4 was now changed from 35.26 g (0.115 mol) to 38.08 g (0.124 mol), and that the amount of anisole added was now changed from 250 g to 200 g. As a result, an aromatic bismaleimide compound represented by the following formula (A-5) was obtained. The number average molecular weight (Mn) of the aromatic bismaleimide compound obtained was 47,500. A varnish was also prepared in a similar manner as the working example 4 after synthesis.

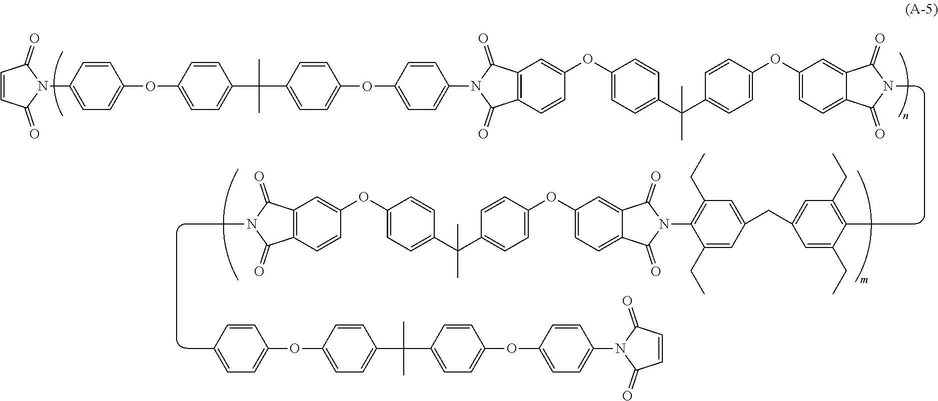

m=25, n=1 (both are average values)

### Comparative Example 4

Added were 16 parts by mass of a linear alkyl group-containing maleimide compound (BMI-3000J, Mn: 6,700 by Designer Molecules Inc.), 0.32 parts by mass of dicumylperoxide and 84 parts by mass of anisole, followed by keeping stirring them under room temperature until they had all dissolved, thereby obtaining a composition.

### Comparative Example 5

A composition was obtained in a similar manner as the comparative example 4, except that the linear alkyl group-containing maleimide compound in the comparative example 4 was now changed to 4,4′-diphenylmethanebismaleimide (BMI-1000, Mn: 410 by Daiwa Fine Chemicals Co., Ltd.).

### Comparative Example 6

A polyamic acid varnish (KJR-655 by Shin-Etsu Chemical Co., Ltd., NMP-containing varnish, non-volatile content 15% by mass) was directly used.

### Comparative Example 7

An amic acid was synthesized by adding 65.06 g (0.125 mol) of 2,2-bis[4-(2,3-dicarboxyphenoxy)phenyl]propane dianhydride, 35.26 g (0.115 mol) of 4,4-methylenebis(2,6-diethylaniline) and 250 g of anisole to a 1 L glass four-necked flask equipped with a stirrer, a Dean-Stark apparatus, a cooling condenser and a thermometer, and then stirring them at 80° C. for three hours. Later, the temperature was directly raised to 150° C., and stirring was performed for another two hours while distilling away a water produced as a by-product, thereby synthesizing a copolymer.

Next, an amic acid was synthesized by adding 7.05 g (0.015 mol) of 2,2-bis[4-(4-aminophenoxy)phenyl]propane to the flask containing the copolymer solution that had been cooled to room temperature, and then performing stirring at 80° C. for three hours. Later, the temperature was directly raised to 150° C., and stirring was performed for another two hours while distilling away a water produced as a by-product, thereby synthesizing a diamine compound with each end being blocked with an amino group.

A maleamic acid was synthesized by adding 1.45 g (0.015 mol) of a maleic anhydride to the flask that had been cooled to room temperature and now contained the solution of the obtained diamine compound, and then performing stirring at 80° C. for three hours. Later, the temperature was directly raised to 150° C., and stirring was performed for another two hours while distilling away a water produced as a by-product, thereby obtaining a varnish of an aromatic bismaleimide compound. This varnish was then heated at 180° C. for 48 hours. The number average molecular weight (Mn) of this aromatic bismaleimide compound was 69,000. Anisole was then added to the varnish in a way such that non-volatile constituents would be in an amount of 16% by mass, followed by adding 2 parts by mass of dicumylperoxide per 100 parts by mass of the non-volatile constituents, and then keeping performing stirring under room temperature until the dicumylperoxide had dissolved, thereby obtaining a composition.

As for each of the compositions obtained in the working examples 4 to 7 and the comparative examples 4 to 7, a solubility thereof in each of the organic solvents shown in Table 2 was evaluated. With regard to the polyamic acid varnish in the comparative example 6, the solubility thereof was evaluated after once removing therefrom NMP as a solvent by heating under a reduced pressure. Further, with regard to each of the compositions, a viscosity thereof was measured after preparing an anisole solution of the composition containing the component (A) by 25% by mass. The viscosity was measured by a method described in JIS K 7117-1:1999, and a rotary viscometer was used to carry out the measurement at 25° C. Here, as for the comparative examples 5 and 6, viscosity measurements were not performed due to an insufficient solubility thereof in anisole. The results are shown in Table 2.

### Production of Cured Product (Film)

Using a roller coater, each of the compositions obtained in the working examples 4 to 7 and the comparative examples 4 to 7 was applied to a PET film having a thickness of 38 μm in a manner such that a thickness of the composition would eventually become 50 μm after drying. Heating was then performed at 130° C. for an hour, and at 180° C. for another two hours to obtain a cured product (film) (curing condition A). Here, in the case of the comparative example 6, since curing was thought to be insufficient with the above curing conditions, heating was performed at 150° C. for an hour, at 200° C. for another hour, and then at 250° C. for yet another four hours to obtain a cured product (film) (curing condition B). Further, in the case of the comparative example 7, the evaluations described below were not conducted, since a cured product (film) failed to be obtained due to poor solvent removal after heating and a failure in removing voids accordingly.

The glass-transition temperature, relative permittivity, dielectric tangent and adhesion force of each of the cured products (films) obtained were measured under the following conditions. The results thereof are shown in Table 3.

### Glass-Transition Temperature

A TMA device (Q400 by TA Instruments) was used to measure the glass-transition temperature of each cured product (film) obtained.

### Relative Permittivity, Dielectric Tangent

A network analyzer (E5063-2D5 by Keysight Technologies) and a stripline (by KEYCOM Corp.) were then connected to each cured product (film) obtained so as to measure the relative permittivity and dielectric tangent thereof at a frequency of 10 GHz.

### Adhesion Force

### Adhesion Force Test Before Moisture Absorption

Each of the compositions obtained in the working examples 4 to 7 and the comparative examples 4 to 7 was sprayed onto a frame substrate prepared by nickel-plating a 20 mm×20 mm copper frame. The composition was then cured under the curing condition(s) shown in Table 3 to form a cured film (primer).

KMC-2110G-7 which is an epoxy resin molding material for semiconductor encapsulation by Shin-Etsu Chemical Co., Ltd. was then molded into a cylindrical shape on the cured film, the cylindrical shape having a base area of 10 mm² and a height of 3 mm (cured under a condition of: pressure 6.9 MPa, temperature 175° C., 120 sec). Later, the test specimen was subjected to post curing at 180° C. for four hours, and a multi-functional bond tester (DAGE SERIES 4000 by Nordson Dage) was then used to measure, at a rate of 0.2 mm/sec, an adhesion force of the post-cured test specimen at room temperature before the test specimen had absorbed moisture.

Adhesion Force Test after Moisture Absorption

In order to measure an adhesion force after moisture absorption, a test specimen was prepared in a similar manner as the adhesion force test before moisture absorption. After being placed in an atmosphere of 85° C./85% RH for 168 hours, the test specimen was then subjected to IR reflow three times at 260° C., followed by using a multi-functional bond tester (DAGE SERIES 4000 by Nordson Dage) to measure, at a rate of 0.2 mm/sec, an adhesion force of the test specimen at room temperature after the test specimen had absorbed moisture.

When there was no cured film (primer), all the epoxy resin molding material was peeled off at the time of performing molding.

TABLE 2

| | | Working | Working | Working | Working | Comparative | Comparative | Comparative | Comparative |
|---|---|---|---|---|---|---|---|---|---|
| | | example 4 | example 5 | example 6 | example 7 | example 4 | example 5 | example 6 | example 7 |
| Solubility | Anisole | >80 | >80 | >100 | >80 | >80 | <5 | <5 | <30 |
| [g/100 g] | Toluene | <60 | <60 | <80 | <40 | >100 | <5 | <5 | <5 |
| | DMF | >100 | >100 | >100 | >100 | >100 | >100 | <20 | <80 |
| | NMP | >100 | >100 | >100 | >100 | >100 | >100 | >100 | <100 |

| | | | | | | | | |
|---|---|---|---|---|---|---|---|---|
| Anisole solution viscosity | 1.2 | 0.9 | 0.2 | 6.4 | 0.3 | — | — | 10.5 |
| (25 wt %)[Pa · s] | | | | | | | | |

TABLE 3

| | | Working | Working | Working | Working | Comparative | Comparative | Comparative | | Comparative |
|---|---|---|---|---|---|---|---|---|---|---|
| | | example 4 | example 5 | example 6 | example 7 | example 4 | example 5 | example 6 | | example 7 |
| Curing condition | | A | A | A | A | A | A | A | B | A |
| Glass-transition | | 200 | 193 | 183 | 223 | 15 | 205 | 175 | 220 | Voids |
| temperature [° C.] | | | | | | | | | | occurred, |
| Relative permittivity | | 2.8 | 2.7 | 2.8 | 2.7 | 2.5 | 3.8 | 3.2 | 3.2 | poor |
| (10 GHz) | | | | | | | | | | solvent |
| Dielectric tangent | | 0.007 | 0.008 | 0.007 | 0.008 | 0.003 | 0.016 | 0.011 | 0.010 | removal |
| (10 GHz) | | | | | | | | | | observed; |
| Adhesion | Before | 28.2 | 29.8 | 26.3 | 28.5 | 18.9 | Peeled | 7.5 | 18.0 | sample |
| force | moisture | | | | | | off when | | | production |
| [MPa] | absorption | | | | | | performing | | | failed |
| | After | 10.5 | 11.5 | 9.3 | 13.6 | 8.8 | molding | 1.2 | 2.1 | |
| | moisture | | | | | | | | | |
| | absorption | | | | | | | | | |

Curing condition A:
(Heated at 130° C. for 1.0 hour)+(Heated at 180° C. for 2.0 hours)

### Curing Condition B:

(Heated at 150° C. for 1.0 hour)+(Heated at 200° C. for 1.0 hour)+(Heated at 250° C. for 4 hours)

### Primer Composition for Copper Substrate

Anisole was added to the component (A) shown in Table 4 in a way such that non-volatile constituents would be in an amount of 16% by mass, followed by adding 2 parts by mass of the component (B) shown in Table 4 per 100 parts by mass of the non-volatile constituents, and then keeping stirring them at room temperature until they had dissolved, thereby obtaining a composition.

The composition obtained was sprayed onto a 20 mm×20 mm copper frame substrate, and was cured under the curing conditions shown in Table 4, thereby obtaining a cured film (primer).

### Initial Adhesion Force Test

KMC-2110G-7 which is an epoxy resin molding material for semiconductor encapsulation by Shin-Etsu Chemical Co., Ltd. was then molded into a cylindrical shape on such cured film, the cylindrical shape having a base area of 10 mm² and a height of 3 mm (cured under a condition of: pressure 6.9 MPa, temperature 175° C., 120 sec). Later, the test specimen was subjected to post curing at 180° C. for four hours, and a multi-functional bond tester (DAGE SERIES 4000 by Nordson Dage) was then used to measure, at a rate of 0.2 mm/sec, an initial adhesion force of the post-cured test specimen at room temperature.

Adhesion Force Test after Heat Treatment

A test specimen was prepared in a similar manner as the initial adhesion force test. After treating this test specimen at 180° C. for 1,000 hours, a multi-functional bond tester (DAGE SERIES 4000 by Nordson Dage) was used to measure, at a rate of 0.2 mm/sec, an adhesion force of the test specimen at room temperature.

TABLE 4

| | | Working | Working | Working | Working | Working | Comparative | Comparative |
|---|---|---|---|---|---|---|---|---|
| | | example 8 | example 9 | example 10 | example 11 | example 12 | example 8 | example 9 |
| (A) | | A-1 | A-1 | A-1 | A-1 | A-3 | A′-1 | A′-2 |
| (B) | | B-1 | B-2 | B-2 | B-2 | B-3 | — | B-2 |
| Curing condition | | A | C | C | A | C | B | A |
| Curing atmosphere | | Nitrogen | Nitrogen | Air | Nitrogen | Air | Air | Nitrogen |
| Adhesion | Initial | 22.5 | 24.6 | 24.5 | 20.2 | 22.6 | All | 16.5 |
| force | | | | | | | peeled off | |
| (MPa) | After heat | 19.6 | 23.5 | 23.2 | — | 22.1 | — | 3.2 |
| | treatment | | | | | | | |

A-1: Aromatic bismaleimide compound obtained in the working example 4
A-3: Aromatic bismaleimide compound obtained in the working example 5
A′-1: KJR-655 (Polyamic acid varnish by Shin-Etsu Chemical Co., Ltd., NMP-containing varnish, non-volatile content 15% by mass)
A′-2: BMI-3000J (linear alkyl group-containing maleimide compound by Designer Molecules Inc., Mn: 6,700)
B-1: Dicumylperoxide (1 hour half-life temperature: 137.5° C.)
B-2: 2-ethylhexanoic acid-t-amyl peroxide (1 hour half-life temperature: 88° C.)
B-3: 1,6-bis(tert-butylperoxycarbonyloxy)hexane (1 hour half-life temperature: 115° C.)

### Curing Condition A:

(Heated at 130° C. for 1.0 hour)+(Heated at 180° C. for 2.0 hours)

### Curing Condition B:

(Heated at 150° C. for 1.0 hour)+(Heated at 200° C. for 1.0 hour)+(Heated at 250° C. for 4 hours)

### Curing Condition C:

(Heated at 110° C. for 1.0 hour)+(Heated at 130° C. for 2.0 hours)

It became clear that when the resin composition of the present invention contains, as a reaction initiator, an organic peroxide having a 1 hour half-life temperature of 80 to 115° C., and is used as a primer for a non-plated copper, the composition can be cured at a low temperature, the copper will not be oxidized, and discoloration at the time of curing can be restricted.

What is claimed is:

1. An aromatic bismaleimide compound represented by the following formula (1): 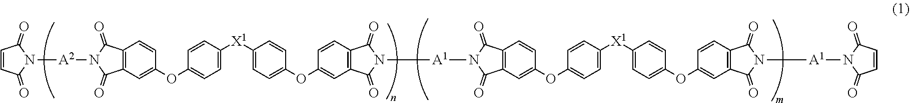
wherein X¹ independently represents a divalent group, each of A¹ and A² independently represents a divalent aromatic group, m represents a number of 1 to 30, n represents a number of 1 to 5,
the divalent group represented by X¹ being selected from groups expressed by the following formulae: 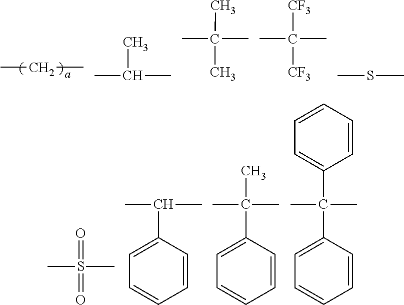
wherein a represents a number of 1 to 6,
the divalent aromatic group represented by each of A¹ and A² being expressed by the following formula (2) or (3): 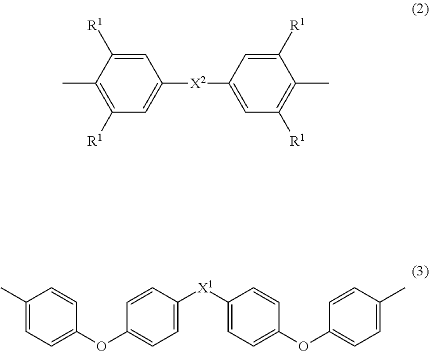
wherein X¹ is defined as above, X² independently represents a divalent group, R¹ independently represents a hydrogen atom, a chlorine atom, or a substituted or unsubstituted aliphatic hydrocarbon group having 1 to 6 carbon atoms, the divalent group represented by X² being selected from groups expressed by the following formulae: 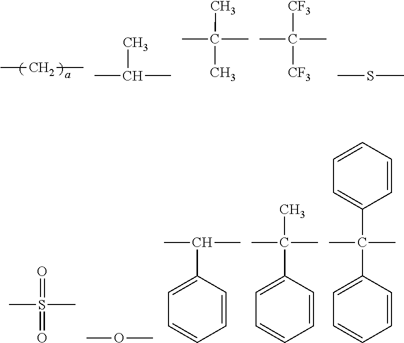
wherein a represents a number of 1 to 6.

2. The aromatic bismaleimide compound according to claim 1, wherein a number average molecular weight of the aromatic bismaleimide compound represented by the formula (1) is 3,000 to 50,000.

3. The aromatic bismaleimide compound according to claim 1, wherein the divalent groups represented by X¹ in the formula (1) and X¹ in the formula (3) are identical to each other.

4. The aromatic bismaleimide compound according to claim 1, wherein in the formula (1), when A¹ is represented by the formula (2), A² is represented by the formula (3); or when A¹ is represented by the formula (3), A² is represented by the formula (2).

5. A method for producing the aromatic bismaleimide compound according to claim 1, comprising:
a step A of synthesizing an amic acid by reacting an aromatic diphthalic anhydride with an aromatic diamine at a molar ratio of aromatic diphthalic anhydride/aromatic diamine=1.01 to 1.50/1.0, and then performing cyclodehydration;
a step B subsequent to the step A, which is a step of synthesizing an amic acid with a reactant obtained in the step A and an aromatic diamine, and then performing cyclodehydration; and
a step C subsequent to the step B, which is a step of synthesizing a maleamic acid by reacting a reactant obtained in the step B with a maleic anhydride, and then performing cyclodehydration to block molecular chain ends with maleimide groups,
wherein the aromatic diphthalic anhydride used in the step A is represented by the following formula (4): 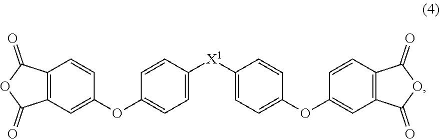
the aromatic diamine used in the step A is represented by the following formula (5): 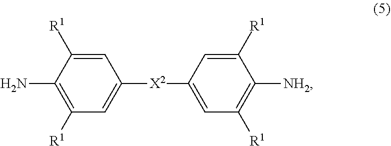
the aromatic diamine used in the step B is represented by the following formula (6): 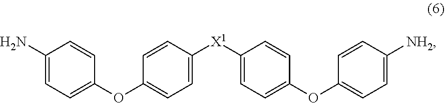
wherein in the formulae (4) and (6), X¹ independently represents a divalent group selected from groups expressed by the following formulae: 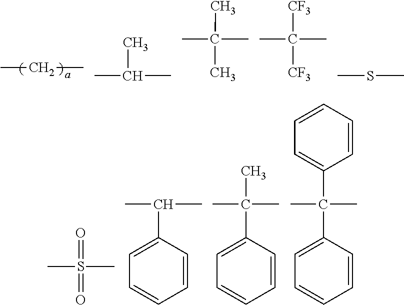
wherein a represents a number of 1 to 6; and
wherein in the formula (5), R¹ independently represents a hydrogen atom, a chlorine atom, or a substituted or unsubstituted aliphatic hydrocarbon group having 1 to 6 carbon atoms, X² independently represents a divalent group selected from groups expressed by the following formulae: 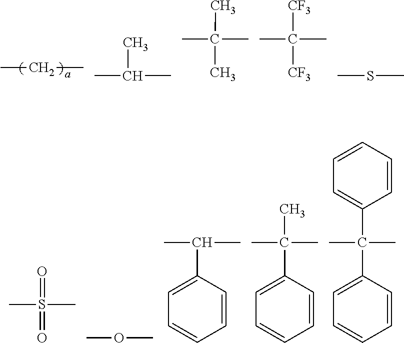
wherein a represents a number of 1 to 6.

6. A method for producing the aromatic bismaleimide compound according to claim 1, comprising:
a step A′ of synthesizing an amic acid by reacting an aromatic diphthalic anhydride with an aromatic diamine at a molar ratio of aromatic diphthalic anhydride/aromatic diamine=1.01 to 1.50/1.0, and then performing cyclodehydration;
a step B′ subsequent to the step A′, which is a step of synthesizing an amic acid with a reactant obtained in the step A′ and an aromatic diamine, and then performing cyclodehydration; and
a step C′ subsequent to the step B′, which is a step of synthesizing a maleamic acid by reacting a reactant obtained in the step B′ with a maleic anhydride, and then performing cyclodehydration to block molecular chain ends with maleimide groups,
wherein the aromatic diphthalic anhydride used in the step A′ is represented by the following formula (4): 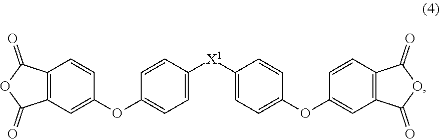
the aromatic diamine used in the step A′ is represented by the following formula (6): 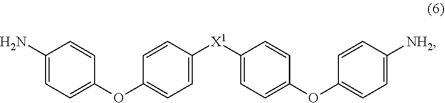
the aromatic diamine used in the step B′ is represented by the following formula (5): 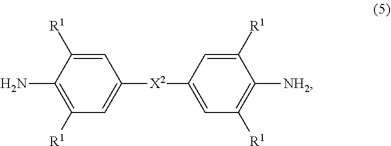
wherein in the formulae (4) and (6), X¹ independently represents a divalent group selected from groups expressed by the following formulae: 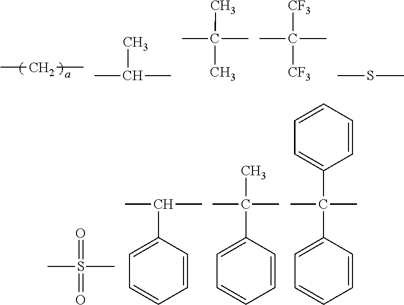
wherein a represents a number of 1 to 6; and
wherein in the formula (5), R¹ independently represents a hydrogen atom, a chlorine atom, or a substituted or unsubstituted aliphatic hydrocarbon group having 1 to 6 carbon atoms, X² independently represents a divalent group selected from groups expressed by the following formulae: 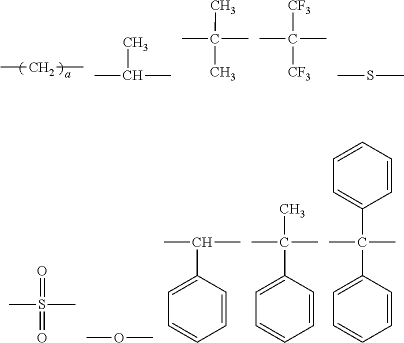
wherein a represents a number of 1 to 6.

7. A heat-curable cyclic imide resin composition comprising:
(A) the aromatic bismaleimide compound according to claim 1;
(B) a reaction initiator; and
(C) an organic solvent.

8. The heat-curable cyclic imide resin composition according to claim 7, wherein the organic solvent (C) is at least one selected from the group consisting of methylethylketone (MEK), cyclohexanone, ethyl acetate, tetrahydrofuran (THF), isopropanol (IPA), xylene, toluene and anisole.

9. The heat-curable cyclic imide resin composition according to claim 7, wherein the reaction initiator (B) has a 1 hour half-life temperature of 80 to 115° C., and the composition is for use as a primer.

10. The heat-curable cyclic imide resin composition according to claim 9, wherein the organic solvent (C) is at least one selected from the group consisting of cyclohexanone, tetrahydrofuran (THF), isopropanol (IPA), xylene, toluene and anisole.

11. A method for producing a cured product, comprising:
curing the heat-curable cyclic imide resin composition according to claim 9 at a temperature of not higher than 150° C.

12. An adhesive agent composition, primer composition, composition for substrate or coating material composition comprising the heat-curable cyclic imide resin composition according to claim 7.

13. A cured product of the heat-curable cyclic imide resin composition according to claim 7.

14. A semiconductor device having the cured product of the heat-curable cyclic imide resin composition according to claim 13.

15. A substrate material having the cured product of the heat-curable cyclic imide resin composition according to claim 13.
